(19) United States
(12) Patent Application Publication
(10) Pub. No.: US 2020/0294656 A1
(43) Pub. Date: Sep. 17, 2020

(54) SCHEDULE MANAGEMENT SYSTEM

(71) Applicant: IRYOU JYOUHOU GIJYUTU KENKYUSYO CORPORATION, Fukuoka (JP)

(72) Inventor: Shinkichi HIMENO, Fukuoka (JP)

(21) Appl. No.: 16/891,728

(22) Filed: Jun. 3, 2020

Related U.S. Application Data
(63) Continuation-in-part of application No. 15/527,164, filed on May 16, 2017.

(30) Foreign Application Priority Data
Nov. 20, 2014 (JP) 2014-235456

(51) Int. Cl. G16H 40/20; G16H 40/40; G06Q 10/06; G06F 3/0482

(52) U.S. Cl. CPC G16H 40/20; G16H 40/40; G06F 3/0482; G06Q 10/06314; G06Q 10/063112

(57) ABSTRACT

Provided is a system with which it is possible for schedule management to be executed efficiently, by making it possible for management of a list of work items required for each patient, management of a list of resources required to implement the work items, and allocation of resources such as people, equipment and locations available for each work item to be performed smoothly, and where possible, by allocating work preferentially to staff familiar with the relevant patient. A schedule management system for creating a schedule of work required for each patient, and for creating an operating schedule for staff, equipment, locations and the like, is provided with: a patient-specific work item list managing means for creating, displaying and editing a list of work items to be carried out for each patient; an allocatable resource list managing means for creating, displaying and editing a resource list of staff, equipment, locations and the like that can be allocated to each work item; and a resource allocating means for each work item, whereby necessary resources from the resource list are allocated for each work item.

### FIELD OF THE INVENTION

The present invention relates to a schedule management system for creating, adjusting and managing a schedule by which a staff such as a nurse and a caregiver gives treatment or care to a patient or a resident in a medical care, a nursing care, and a welfare agency or the like, and more specifically to a schedule management system for creating a schedule of a work plan for each patient or user, and properly allocating an article, a place and the like required by staff for each plan so that various medical cares and caring works conducted by a plurality of staffs comprising a plurality of occupations would smoothly proceed for a plurality of users in a hospital, a welfare facility, a nursing home and the like.

### BACKGROUND OF THE INVENTION

In a medical institution such as a hospital, a detailed treatment schedule is made out in accordance with a disorder for each patient. The schedule includes drip infusion, change of bandage, rehabilitation, toilet and dining assists, position change and the like. Even in a nursing care, and a welfare agency, although there is less medical procedure, there are many caring actions such as assists of toilet, dining and bathing, diaper changing, position change for preventing pressure ulcer and recreation, after all, a detailed caring schedule called care plan is created.

As a scheme to create a schedule of medical practice, a clinical pathway has been proposed (Patent Document 1).

### CITATION LIST

### Patent Literature

[Patent Document 1] JP No. 2006-113795 A

This is to prepare, for each disorder, a form of a schedule for what drip infusion or treatment should be performed on which day after being hospitalized, and to develop a treatment schedule on an electronic medical record in accordance with the disorder of a patient who has been hospitalized and a date of admission. By this scheme, a treatment schedule standardized for each disorder is automatically developed, and thus an efficient work can be conducted, in which there is no failure of instruction and no overlapping of a work item, and the scheme is becoming popular (FIG. 1).

### SUMMARY OF THE INVENTION

### Problem to be Solved by the Invention

In the aforementioned clinical pathway, although a work item list for each patient and each day can be obtained, in order to perform a work, it is necessary to allocate a time at which the work is actually performed, a staff, equipment and place for each work item. There are cases where a plurality of staffs are required depending a work item. In a case where a place and equipment are limited such as bathing, it is necessary to adjust a using order among patients. In addition, it is desirable that a staff with whom a patient is familiar is preferably in charge of a work for the patient. Conventionally, such allocation work has been done manually on paper by an experienced manager such as a ward chief or head nurse. The work is related to many factors, and thus is not only time-consuming, but also generating many allocation mistakes.

The manager can manage to make an adjustment within the same occupation such as nurse; however, when many occupations such as rehabilitation and caring are related, the adjustment would be more difficult. As a matter of fact, there are often unworkable problems due to such an interference of time, resources or the like, that rehabilitation cannot be conducted because when a rehabilitation therapist visits a room for rehabilitation, there are times when a nurse is performing a drip infusion work, or a caregiver is taking a patient out for bathing. Meanwhile, when a work is canceled by physical deconditioning or the like, a staff who is in charge of the work cannot easily grasp a work schedule of other alternative patient, and thus the staff often idly spends time doing nothing, which is a cause of a decline in work efficiency.

A staff must perform various works of medical care, nursing and caring on assigned plurality of patients. It is especially difficult to accurately perform a specified work for a specified patient at a specified time. Although spending too much labor for confirmation, unfortunately, there are quite a lot of malpractices such as omission of performing and duplicative performing and a mix-up of a patient. In addition, it is necessary to create a performance record after performing a work, and the recording work also takes time. As a treatment proceeds, content of a work for each patient changes. Moreover, because of shift work, there is no guarantee that a same staff can be allocated. For these reasons, it is necessary to repeat a resource allocation work from scratch every day, and a huge amount of work by managers with a high hourly rate is required.

The present invention was made to solve such conventional problem, and it is an object of the invention to provide a system which can perform an efficient schedule management through such as: smoothly performing a management of a work item list required for each patient, a management of a list of resources required for performing a work item and an allocation of resources such as an available person, equipment and place for each work item; preferably allocating a work preferentially to a staff with whom a patient is familiar; if there is an interference of time, person, equipment, place or the like, detecting the interference and making an adjustment; rescheduling to reduce idle time upon cancellation; supporting a field staff so that he/she can easily and accurately grasp his/her own work schedule, perform same for a patient without fault and easily keep a record; and optimizing the reuse of a created work schedule, thereby reducing labor for creating same.

### Means for Solving the Problem

As a means to achieve the aforementioned objective, in the schedule management system according to claim 1 of the present invention, a schedule management system for creating a work schedule required for each patient and an operation schedule of a staff, equipment, and a place is provided with: a patient-specific work item list managing means for creating, displaying and editing a list of work items of a plurality of occupations that should be performed for each patient; an allocatable resource list managing means for creating, displaying and editing a resource list of a staff, equipment, and a place of a plurality of occupations, that are allocatable for each work item; and a per work item resource allocating means for allocating a required resource from the aforementioned resource list for the each work item. The aforementioned per work item resource allocating means is provided with a function to allocate the resource by displaying in parallel a patient-specific work item list and an allocatable resource list, and dragging & dropping a work item object to an allocatable resource object.

In the schedule management system according to claim 2, in the schedule management system according to claim 1, the aforementioned per work item resource allocating means is provided with a staff duty roster managing means for separately creating and managing a staff duty roster, and enabling to specify a staff on duty on each day and for whom a resource allocation is possible, and displaying only an allocatable resource list of an allocatable staff when allocating resources required for performing said work item.

In the schedule management system according to claim 3, in the schedule management system according to either one of claim 1 or 2, the aforementioned per work item resource allocating means is provided with a resource allocation verification means for verifying whether or not the resource required for performing said work item is allocated, whether or not the resource overlaps with a resource and a time allocated for other work item, and if there is no problem for performing said work item.

In the schedule management system according to claim 4, in the schedule management system according to any one of claims 1 to 3, a schedule duplicating means for duplicating, a part or the whole of the aforementioned work schedule required for each patient and the operation schedule of a staff, equipment and a place, created by the schedule management system, in another day of the same patient, or in another patient is provided.

In the schedule management system according to claim 5, in the schedule management system according to any one of claims 1 to 4, the aforementioned patient-specific work item list managing means is provided with a work item type-specific required resource setting means for setting in advance, by type of the work item, a resource required for performing the work such as an occupation and a number of required staffs, required equipment and a place for performing the work.

In the schedule management system according to claim 6, in the schedule management system according to any one of claims 1 to 5, the aforementioned patient-specific work item list managing means is provided with an electronic medical record to work item list importing means for creating a work item list required for a patient by importing same from a work instruction list of an electronic medical record of said patient, and a work item list to electronic medical record writing back means for writing the work item list done with a resource allocation back to the electronic medical record.

In the schedule management system according to claim 7, in the schedule management system according to any one of claims 1 to 6, the aforementioned patient-specific work item list managing means is provided with a performed work record creating means for creating a performed work record with regard to a performed work item.

In the schedule management system according to claim 8, in the schedule management system according to claim 7, the aforementioned patient-specific work item list managing means is provided with a performed work record writing back to electronic medical record means for writing the aforementioned created performed work record back to a patient medical record.

In the schedule management system according to claim 9, in the schedule management system according to claim 7 or 8, the aforementioned performed work record creating means is provided with a patient ID comparing means for comparing a patient ID display means carried by a patient with a patient ID specified for said work item, and confirming that both IDs match.

In the schedule management system according to claim 10, in the schedule management system according to any one of claims 1 to 9, the aforementioned per work item resource allocating means is provided with a preferred resource allocation per-patient and per-work item type setting means for setting a resource that should be allocated preferentially for said patient for an each patient and an each work item type.

### Advantages of the Invention

In the schedule management system according to claim 1, the patient-specific work item list managing means for creating, displaying and editing a list of work items that should be performed for each patient is provided, and thus a work required for each patient is clearly grasped. In addition, the allocatable resource list managing means for creating, displaying and editing a resource list such as a staff, equipment, and a place, that are allocatable for each work item is provided, and thus an available resource is properly managed in advance. Furthermore, the per work item resource allocating means for allocating a required resource from the aforementioned resource list for the each work item is provided, and thus a required resource corresponding to a work can be properly allocated.

In the schedule management system according to claim 2, the staff duty roster managing means for separately creating and managing a staff duty roster, and enabling to specify a staff on duty on each day and for whom a resource allocation is possible, and displaying only an allocatable resource list of an allocatable staff when allocating resources required for performing said work item is provided, and thus a selection of a staff, a creation and a management of a work list can be performed easily and accurately.

In the schedule management system according to claim 3, the resource allocation verification means for verifying if there is no problem for performing said work item, such as that whether or not a resource required for performing a work item is allocated, and whether or not the resource overlaps with a resource and a time allocated for other work item is provided, and thus unenforceability due to lack of or overlapping of resources is prevented.

In the schedule management system according to claim 4, the schedule duplicating means for duplicating, a part or the whole of the created work schedule required for each patient and the operation schedule of staff, equipment and place, in another day of the same patient, or in another patient is provided, and thus a once created work schedule can be reused wherever possible, and efficiency in inputting can be thereby achieved.

In the schedule management system according to claim 5, the work item type-specific required resource setting means for setting in advance, by type of the work item, a resource required for performing the work such as an occupation and a number of required staffs, required equipment and a place for performing the work is provided, and thus, only by designating a work item, a required resource is specified, and a corresponding candidate is listed up.

In the schedule management system according to claim 6, the electronic medical record to work item list importing means for creating a work item list required for a patient by importing same from a work instruction list of an electronic medical record of the patient, and the work item list to electronic medical record writing back means for writing the work item list done with a resource allocation back to the electronic medical record are provided, and thus a work item is written back to an electronic medical record and united, and a work performed, an observation record and the like for a patient are integrally record and can be referred to.

In the schedule management system according to claim 7, the performed work record creating means for creating a performed work record with regard to a performed work item is provided, and thus whether or not a work has been performed can be easily confirmed, and omission of performing and duplicative performing is prevented.

In the schedule management system according to claim 8, the performed work record writing back to electronic medical record means for writing the created performed work record back to a patient medical record is provided, and thus a schedule of a work and the performance are securely recorded as a pair on the medical record of the patient.

In the schedule management system according to claim 9, the patient ID comparing means for comparing a patient ID display means carried by a patient with a patient ID specified for said work item, and confirming that both IDs match is provided, and thus a patient is thoroughly checked in accordance with a treatment, and a mix-up of a target patient and creation of an imaginary performance record are prevented.

In the schedule management system according to claim 10, the preferred resource allocation per-patient and per-work item type setting means for setting a resource that should be allocated preferentially for said patient for an each patient and an each work item type is provided, and thus a treatment emphasizing on a human relationship such that a same nurse or a same caregiver are preferentially in charge of a same patient is possible.

### BRIEF DESCRIPTION OF THE DRAWINGS

FIG. 1 is one example of clinical pathway pertaining to a conventional example, and is a diagram showing lists of works that are daily necessary for a patient.

FIG. 2 is an explanatory diagram showing work item lists necessary for two patients per day.

FIG. 3 is an explanatory diagram of a resource allocation example in a caregiver schedule.

FIG. 4 is an explanatory diagram of an object of a concerned work item (toileting assistance).

FIG. 5 is an explanatory diagram of an object of a concerned work item (bathing assist).

FIG. 6 is an explanatory diagram of a resource allocation example in a nurse schedule.

FIG. 7 is an explanatory diagram showing a setting example of a preferentially allocated resource.

### DETAILED DESCRIPTION

### Example

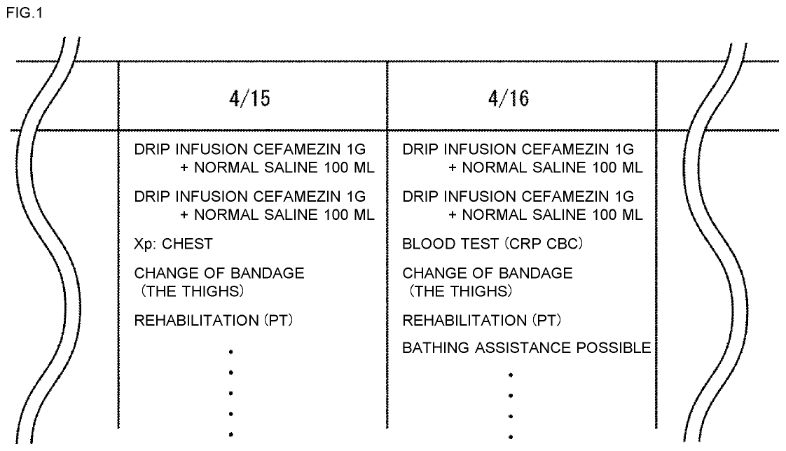

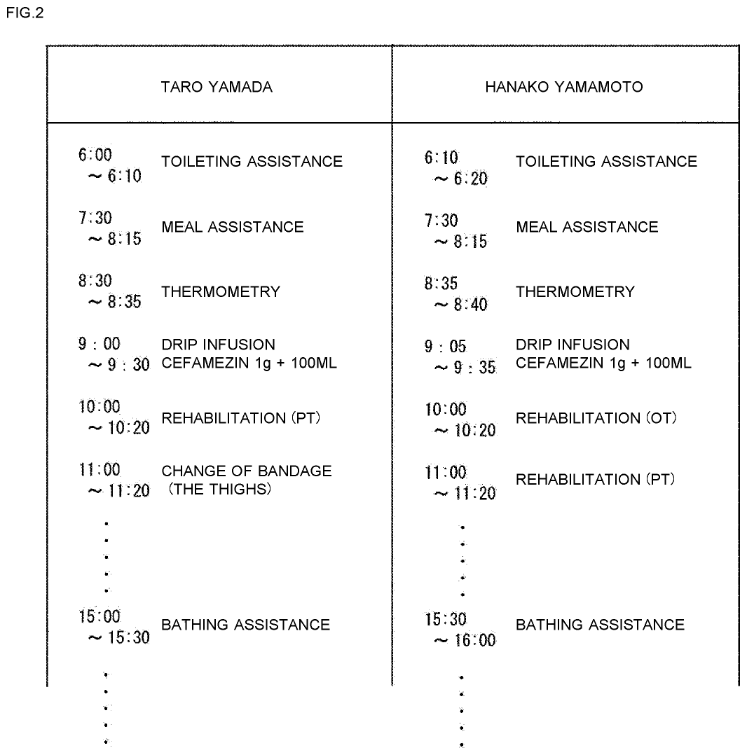

The patient-specific work item list managing means of the present invention will be described with the use of FIG. 2. Different from a conventional clinical pathway of FIG. 1, a work schedule of a day including, for each patient, when each work item is performed, its start time and end time, is tabulated in detail (patient-specific work item list managing means). Looking at this patient-specific work item list, the schedule of the day of said patient can be easily grasped. The work item is respectively assigned to staffs of various occupations such as a nurse, a caregiver, a rehabilitation therapist. In the creation of this patient-specific work item list, a work instruction list as shown in FIG. 1, which is created by a clinical pathway or the like of an electronic medical record is imported, and a time zone is allocated to each work item to create an actual work schedule, whereby a transcription work is eliminated, and thus time is saved, and at the same time, consistency with electronic medical record is ensured (electronic medical record to work item list importing means).

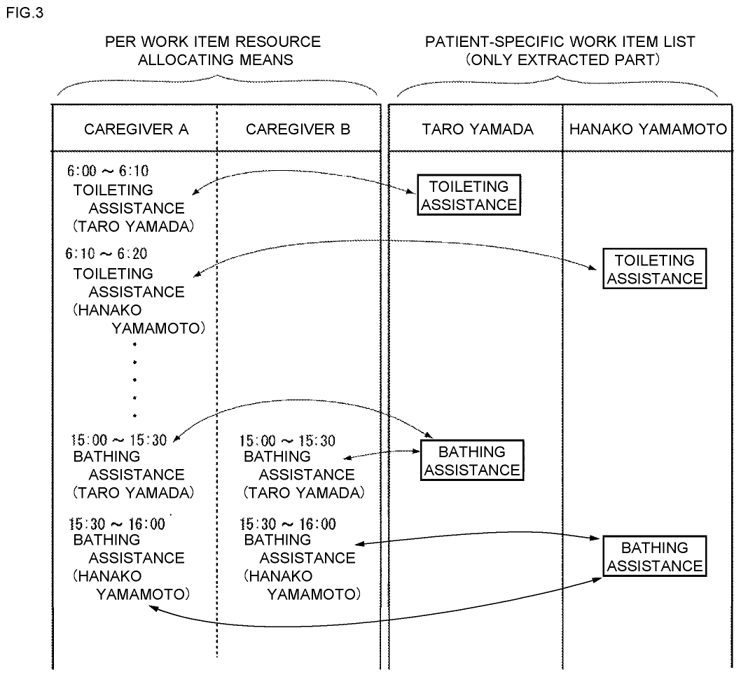

The allocatable resource list managing means and the per work item resource allocating means of the present invention will be described with the use of FIG. 3. The right half of FIG. 3 is such that two work items: toileting assistance and bathing assistance are extracted from the patient-specific work item list of FIG. 2 for purposes of illustration. After confirming that a caregiver A and a caregiver B are on duty on the day from a duty roster (staff duty roster managing means), they are displayed as an allocatable resource list. The duty roster of staffs may be in an arbitrary format as long as a name and an occupation of a staff on duty for each date are recorded. The left half of FIG. 3 shows a schedule of each day of the caregiver A and caregiver B as an allocatable time resource as an example of the allocatable resource list. The caregiver A is in charge of a toileting assistance of a patient, Taro Yamada, at 6:00-6:10 and a patient, Hanako Yamamoto, at 6:10-6:20. This is because that there is no already allocated work item during 6:00-6:20 in the time resource of the caregiver A.

In the per work item resource allocating means of the present invention, an easy-to-use resource allocation operation is achieved with the use of a technique of object oriented programming. Among the work item objects included in said patient-specific work item list, an object of a “concerned work item” (in this case, toileting assistance) is specified by highlighting same by an operation such as a mouse click.

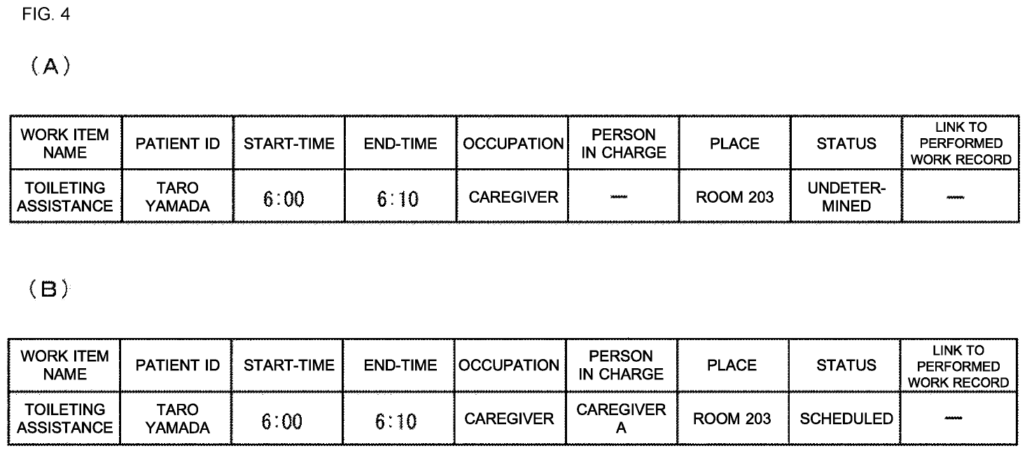

FIG. 4 (A) shows a state of the object of the concerned work item before a resource allocation. Items of resources or the like required for performing a work such as an occupation and a number of required staffs, required equipment, a place for performing and the like are set by type of a work item in advance with the use of a work item type-specific required resource setting means. Although an occupation in charge of the “toileting assistance” work is a caregiver, at the beginning, a column of a caregiver in charge is blank, and a status item indicating the state of the object is “undetermined”. Objects of allocatable resource list of staffs who have an occupation (in this example, caregiver) in charge of said work item and is on duty on the day are displayed in parallel (in this example, two persons), and a staff with free time resource in a time zone corresponding to the aforementioned concerned work item object is selected, and the aforementioned concerned work item object is dragged & dropped to an object of the allocatable resource list of the staff with the aforementioned free time resource, thereby enabling resource allocation. That is, when the aforementioned drag & drop operation is performed, the aforementioned work item object as shown in FIG. 4(A) changes to that as shown in FIG. 4 (B). The caregiver A is entered in a column of the caregiver in charge, and the status item changes to “scheduled”. The completed work item object is stored in said patient-specific work item list and the allocatable resource list of said staff.

When a time zone of an work item object is slightly overlapped, a start-time and an end-time in said object is changed by dragging an object desired be moved, and be adjusted so as to be in an appropriate time zone. When a nurse may be applied as an occupation in charge of a work instead of a caregiver, a plurality of occupations are specified. In this way, when a work item object is highlighted, an object of an allocatable resource list of staffs of an assigned occupation can be automatically displayed in parallel. In addition, all elements in the present invention are objectified, and thus a simple and intuitive operation is enabled; however, a partial operation may be performed with a function key, a pull down menu or the like.

Referring back to FIG. 3, a resource allocation is performed with regard to a work item of bathing assist. Two caregivers are in charge of the bathing assist. The resource allocation can be performed by respectively dragging & dropping a work item object of bathing assist, which is present in a patient-specific work item list object, to the allocatable resource list object of the two caregivers. When performance of the work item is completed, it is necessary to keep a performance record.

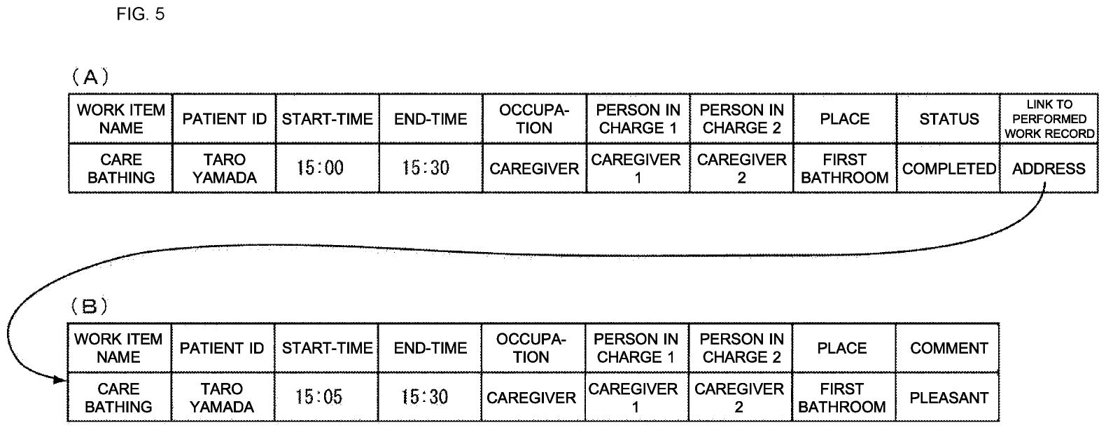

FIG. 5 (A) is the content of the bathing work object. The status is changed to “completed”, and a link to a performed work record is attached. A real form of the link may be an arbitrary format as long as being a format that can specify the performed work record such as an address on a memory or a file and an URL. On the linked object, the performed work record (FIG. 5 (B)) is created. Although a scheduled start-time is 15:00, it is found that an actual start-time is 15:05. A comment for the work can be kept as needed. By this performed work record, at what time who performed a work is kept as a definite record, it is useful to prevent a malpractice such as forgetting of a work, or conversely, duplicative performing.

When performing a work item, a patient ID comparing means for comparing a patient ID obtained from a patient ID display means such as a bar code and an IC chip carried by a patient in a form such as a wristband and a pendant with a patient ID specified for said work item, and confirming that both IDs match is provided, a mix-up of a patient subjected to the work would not occur, and creation of an imaginary performed work record becomes difficult; therefore, credibility of the performed work record is improved. The created work item object and the performance record thereof may be managed only in the schedule management system of the present invention, however, if being written back in an electronic medical record of said patient and united, the work performed, observation record and the like for said patient can be integrally recorded and referred, and thus are useful.

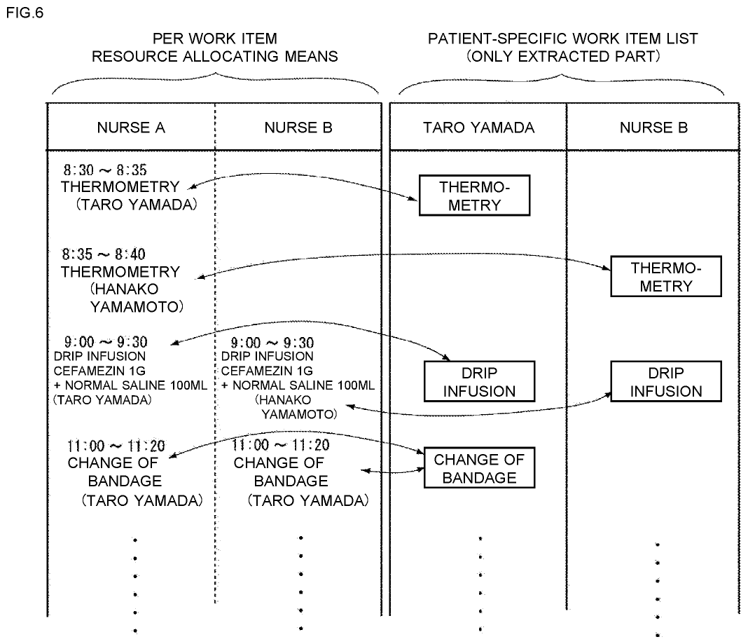

FIG. 6 is a resource allocation example of a nurse to be in charge with regard to a work item such as thermometry and drip infusion. As stated above, a staff performs a work item in accordance with an own allocatable resource list, and creates a performed work record, and thus even if there are a plurality of own patients to be nursed, the staff can perform mixed work items without fault. In addition, a daily workflow is visualized, and thus for a time in which loads are concentrated, a part of work items are shared with other staff, whereby works without an unevenness and an overburden become possible.

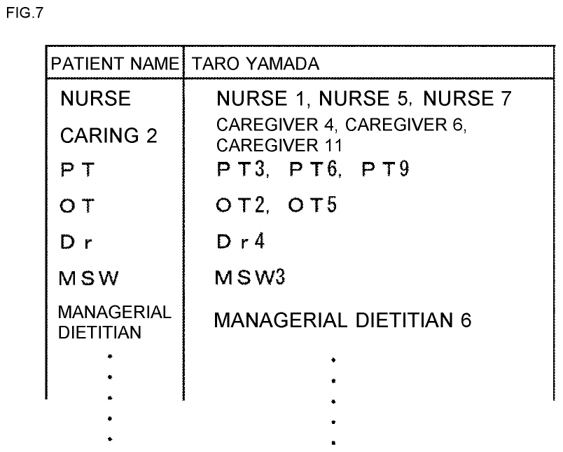

It is known that it is desirable that a same nurse and a caregiver are preferably in charge of a same patient. This is because the patient wants to receive nursing and caring by a reliable staff having a human relationship. FIG. 7 shows a setting example of a preferentially allocated resource for a patient. On condition that a resource to be preferentially allocated by patient is set in advance with the use of a preferentially allocated resource setting means, when a concerned work item object is specified in patient-specific work item list objects as shown in, for example, FIG. 3 or 6, in the allocatable resource list displayed in parallel in the left side of FIGS. 3 and 6, even in a case of a same assigned occupation, an allocatable resource list of a staff a resource of who should be preferentially allocated is preferentially displayed. Although the allocation is performed within the range thereof, if there is no free resource, the range of the allocatable resource list to be shown is extended over a staff having a same occupation other than the preferred allocation.

In a case where only one person is allocated when a plurality of staffs are needed, time zones of work items are overlapped in an allocatable resource list of a staff in charge, and there is an overlapping in an time zone in an allocatable resource list of a limited resource such as a bathroom and test equipment, implementation of an actual work becomes difficult. By using a per work item resource allocation verification means for verifying a problem such as the lack in the resource to be allocated and overlapping of a time zone, a problem can be easily found, and fulfillment of lacking resource and adjustment of the time zone of a work item object are performed, and thus an operable work schedule can be created.

A list of work items that should be performed by patient changes on a daily basis. In addition, staffs also work by shift, and thus a staff on duty also changes every day. For this reason, it is necessary to update a work schedule every day. However, it takes too much time and energy to repeat the works stated above from scratch every day. Therefore, it is important to reuse a once-created work schedule wherever possible. In a schedule duplicating means of the present invention, in a patient-specific work item list of an arbitrary patient, a set of arbitrary work item list objects can be duplicated on an arbitrary day. All work items may be subject to the duplication, and a partial set such as work items of only caring, only rehabilitation, or only nursing may be possible.

In addition, a specific one day may be subject to the duplication, or one week may be duplicated all at once. If, not only the patient-specific work item list, but work item objects of an allocatable resource list of a staff in charge of each work item of said patient-specific work item list are duplicated together, a resource allocation work can be omitted. In doing so, if there is a staff who is not on duty on the day, it is necessary to perform a resource allocation for other staff on duty on the day. A work item that needs an additional resource allocation is detected with the use of the aforementioned per work item resource allocation verification means, and a resource allocation is performed only on the detected part for the staff on duty on the day.

While the Example has been described above, the specific configuration of the present invention is not limited to the aforementioned Example, and a design change or the like without departing from the scope of the invention is included in the present invention. For example, although a hospital is taken as an example here, a similar schedule management system can be constructed for residents of a nursing home and a welfare facility. In addition, the electronic medical record and the schedule management system of the present invention have been described separately for convenience of explanation, the system may be incorporated into a function of the electronic medical record and operated integrally.

In an embodiment, the schedule management system comprises a computer including a processor connected to a memory, In an embodiment, the schedule management system comprises computers including a processor connected to a memory. In an embodiment, the computers comprise a server and a client. The computer includes the processor as an arithmetic device, the memory as a main storage device, an auxiliary storage device, an input device, an output device, a communication device, and a bus interface for interconnection. The computer may adopt a hardware configuration of generic computing devices (generic computers). In an embodiment, the client plays a role of inputting, dragging and dropping the examples of the information.

The processor included in the computer performs the examples of the unit/means/step/function including patient-specific work item list managing means, allocatable resource list managing means, per work item resource allocating means, staff duty roster managing means, resource allocation verification means, schedule duplicating means, patient-specific work item list managing means, work item type-specific required resource setting means, work item list importing means, electronic medical record writing back means, performed work record creating means, electronic medical record means, patient ID comparing means, and preferred resource allocation per-patient and per-work item type setting means.

What is claimed is:

1. A schedule management system for creating a work schedule required for each patient and an operation schedule of a staff, equipment, and a place, the system includes a processor that:
performs creating, displaying and editing a list of work items of a plurality of occupations that should be performed for each patient;
performs creating, displaying and editing a resource list of a staff, equipment, and a place of a plurality of occupations, that are allocatable for each work item;
performs allocating a required resource from the resource list for the each work item;
performs with a function to allocate the resource by displaying in parallel a patient-specific work item list and an allocatable resource list, and dragging & dropping a work item object to an allocatable resource object;
performs highlighting the work item objects included in said patient-specific work item list by a mouse click;
performs displaying an object of said allocatable resource list of staffs of an assigned occupation in parallel when the work item object is highlighted.

2. The schedule management system according to claim 1, wherein the processor separately performs creating and managing a staff duty roster, and enabling to specify a staff on duty on each day and for whom a resource allocation is possible, and displaying only an allocatable resource list of an allocatable staff when allocating resources required for performing said work item.

3. The schedule management system according to claim 1, wherein the processor performs verifying whether or not the resource required for performing said work item is allocated, whether or not the resource overlaps with a resource and a time allocated for other work item, and if there is no problem for performing said work item.

4. The schedule management system according to claim 1, the processor performs duplicating, a part or the whole of the work schedule required for each patient and the operation schedule of a staff, equipment and a place, created by the schedule management system, in another day of the same patient, or in another patient is provided.

5. The schedule management system according to claim 1, wherein the processor performs setting in advance, by type of the work, wherein the resource includes an occupation and number of required staff, required equipment and a place for performing the work.

6. The schedule management system according to claim 1, wherein the processor performs creating a work item list required for a patient by importing same from a work instruction list of an electronic medical record of said patient, and a work item list for writing the work item list done with a resource allocation back to the electronic medical record.

7. The schedule management system according to claim 1, wherein the processor performs creating a performed work record with regard to a performed work item.

8. The schedule management system according to claim 7, wherein the processor performs writing the created performed work record back to a patient medical record.

9. The schedule management system according to claim 7, wherein the processor performs comparing a patient ID carried by a patient with a patient ID specified for said work item, and confirming that both IDs match.

10. The schedule management system according to claim 1, wherein the processor performs setting a resource that should be allocated preferentially for said patient for an each patient and an each work item type.
